(19) United States
(12) Patent Application Publication
(10) Pub. No.: US 2025/0276170 A1
(43) Pub. Date: Sep. 4, 2025

(54) CIRCULATORY SUPPORT DEVICES, SYSTEMS, AND METHODS

(71) Applicant: Boston Scientific Scimed, Inc., Maple Grove, MN (US)

(72) Inventors: Judith Tiferes Wang, Shoreview, MN (US); Arden Michael Thomas Costello, Maple Grove, MN (US); Jennifer Gibbs, White Bear Township, MN (US)

(73) Assignee: Boston Scientific Scimed, Inc., Maple Grove, MN (US)

(21) Appl. No.: 19/065,616

(22) Filed: Feb. 27, 2025

Related U.S. Application Data
(60) Provisional application No. 63/559,362, filed on Feb. 29, 2024.

(51) Int. Cl. A61M 60/585; A61M 60/508

(52) U.S. Cl. CPC A61M 60/585; A61M 60/508; A61M 2205/3365; A61M 2205/3569; A61M 2205/3592; A61M 2205/502; A61M 2205/8206; A61M 2205/8237; A61M 2209/088

(57) ABSTRACT

A circulatory support system may include a blood pump and a removable controller configured to control operation of the blood pump and be removably coupled to a console. The removable controller comprising a display configured to display status indicators corresponding to parameters related to operation of the blood pump; speed controls configured to control a speed of the blood pump; a first connector configured to communicatively couple the removable controller with the blood pump; and a second connector configured to communicatively couple the removable controller with the console.

### CROSS REFERENCE TO RELATED APPLICATIONS

The present application claims the benefit of U.S. Provisional Patent Application Ser. No. 63/559,362, filed Feb. 29, 2024, the disclosure of which is incorporated herein by reference.

### TECHNICAL FIELD

The present disclosure pertains to mechanical circulatory support devices, systems, and methods. More specifically, the present disclosure relates to percutaneous ventricular assist device (PVAD) systems with removable controllers.

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, circulatory assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing, display, operation and optimization of medical devices and systems.

### BRIEF SUMMARY

This disclosure provides design, material, manufacturing method, and use alternatives for medical devices, including ventricular assist devices.

A first example is a circulatory support system. The circulatory support system includes a blood pump and a removable controller configured to control operation of the blood pump and be removably coupled to a console. The removable controller includes a display configured to display status indicators corresponding to parameters related to operation of the blood pump, speed controls configured to control a speed of the blood pump, a first connector configured to communicatively couple the removable controller with the blood pump, and a second connector configured to communicatively couple the removable controller with the console.

Alternatively or additionally to any of the examples herein, in another example, the first connector is disposed at a first surface of the removable controller, and the second connector is disposed at a second surface of the removable controller.

Alternatively or additionally to any of the examples herein, in another example, the first surface is a first side surface and the second surface is a second side surface that is opposite the first side surface.

Alternatively or additionally to any of the examples herein, in another example, the removable controller is configured to be disposed at least partially within a docking port of the console.

Alternatively or additionally to any of the examples herein, in another example, the removable controller is configured to be disposed entirely within the docking port of the console.

Alternatively or additionally to any of the examples herein, in another example, the first surface is coplanar with an outer surface of the console when the removable controller is disposed entirely within the docking port of the console.

Alternatively or additionally to any of the examples herein, in another example, the removable controller is configured such that at least the first connector remains physically accessible when the removable controller is removably coupled to the console.

Alternatively or additionally to any of the examples herein, in another example, at least the first connector and the display remain accessible when the removable controller is removably coupled to the console.

Alternatively or additionally to any of the examples herein, in another example, the removable controller is configured to be in wired communication, wireless communication, or both, with the console.

Alternatively or additionally to any of the examples herein, in another example, the removable controller includes a rechargeable battery.

Alternatively or additionally to any of the examples herein, in another example, the removable controller is configured to receive wireless power, wired power, or both, to recharge the rechargeable battery.

Alternatively or additionally to any of the examples herein, in another example, the system includes a wearable device configured to receive the removable controller. The removable controller is configured to be removably coupled to the wearable device.

Alternatively or additionally to any of the examples herein, in another example, the removable controller includes a third connector disposed on an outer surface of the removable controller. The third connector is configured to removably couple the removable controller to the wearable device.

Alternatively or additionally to any of the examples herein, in another example, the removable controller is configured to remain communicatively coupled to the blood pump when the removable controller is removably coupled to the console to operate the blood pump.

Alternatively or additionally to any of the examples herein, in another example, the removable controller is configured to remain communicatively coupled to the blood pump when the removable controller is decoupled from the console to operate the blood pump.

Another example is a circulatory support system. The circulatory support system includes a blood pump, a console, and a removable controller. The console includes a display configured to display at least a first set of status indicators corresponding to parameters related to operation of the blood pump, a docking port, and a connector. The removable controller includes a user interface configured to display a second set of status indicators corresponding to parameters related to operation of the blood pump, speed controls configured to control a speed of the blood pump, a first connector configured to communicatively couple the removable controller with the blood pump, and a second connector configured to connect to the connector of the console when the removable controller is docked with the console via the docking port to communicatively couple the removable controller to the console.

Alternatively or additionally to any of the examples herein, in another example, the docking port is configured to obscure at least the display of the removable controller when the removable controller docked with the console via the docking port.

Alternatively or additionally to any of the examples herein, in another example, the removable controller is configured to cease display of the second set of status indicators when communicatively coupled to the console.

Alternatively or additionally to any of the examples herein, in another example, the display of the removable controller remains exposed when the removable controller is docked with the console via the docking port and the removable controller is configured to continue to display the second set of status indicators when communicatively coupled to the console.

Another example is a circulatory support system. The circulatory support system includes a blood pump, a console, and a removable controller. The console includes a display configured to display at least a first set of status indicators corresponding to parameters related to operation of the blood pump, a docking port, a connector, and a first battery. The removable controller includes a display configured to display a second set of status indicators corresponding to parameters related to operation of the blood pump, speed controls configured to control a speed of the blood pump, a first connector configured to communicatively couple the removable controller with the blood pump, a second connector configured to connect to the connector of the console when the removable controller is docked with the console via the docking port to communicatively couple the removable controller with the console, and a second battery.

Alternatively or additionally to any of the examples herein, in another example, the removable controller is configured to maintain operation of the blood pump when undocked from the console.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1A is a schematic diagram of an illustrative circulatory support system with a blood pump positioned in a patient;

FIG. 1B is a schematic diagram of another illustrative circulatory support system with a blood pump positioned in a patient;

FIG. 2 is a schematic diagram of an illustrative computing system including a removable controller;

FIG. 3 is a schematic diagram of a portion of an illustrative circulatory support system with a removable controller coupled to a console;

FIG. 4 is a schematic diagram of a portion of an illustrative circulatory support system with the removable controller decoupled from the console;

FIG. 5 is a schematic diagram showing a plurality of surfaces of the removable controller;

FIG. 6 is a schematic diagram showing a removable controller coupled to a patient; and

FIG. 7 is a schematic diagram of a portion of another illustrative circulatory support system with a removable controller coupled to a console.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term

“about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “another configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory support devices and systems are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support devices may support a patient during percutaneous coronary interventions and/or other procedures.

Example cardiac circulatory support devices include, but are not limited to, ventricular assist devices (VADs), total artificial hearts, intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. As a PVAD may be placed at a desired location of anatomy of a patient via percutaneous access and delivery, the PVAD may be used in emergency medicine, a catheter laboratory, and/or other surgical and/or non-surgical settings.

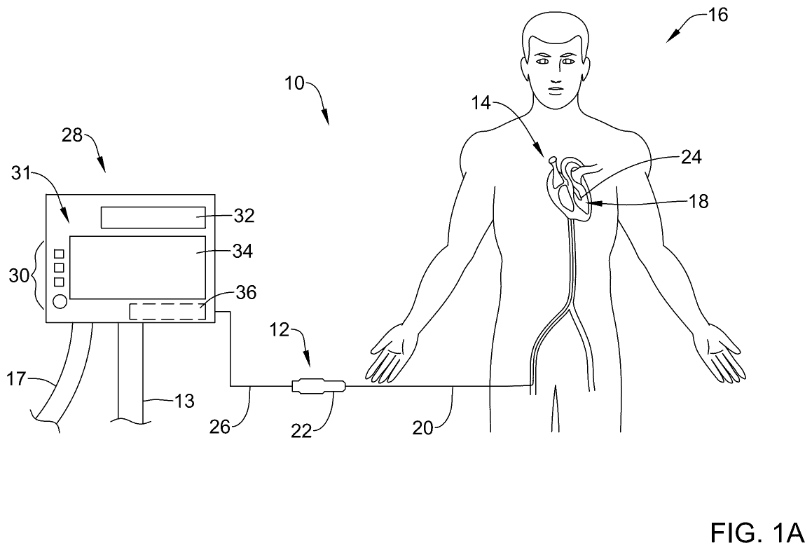

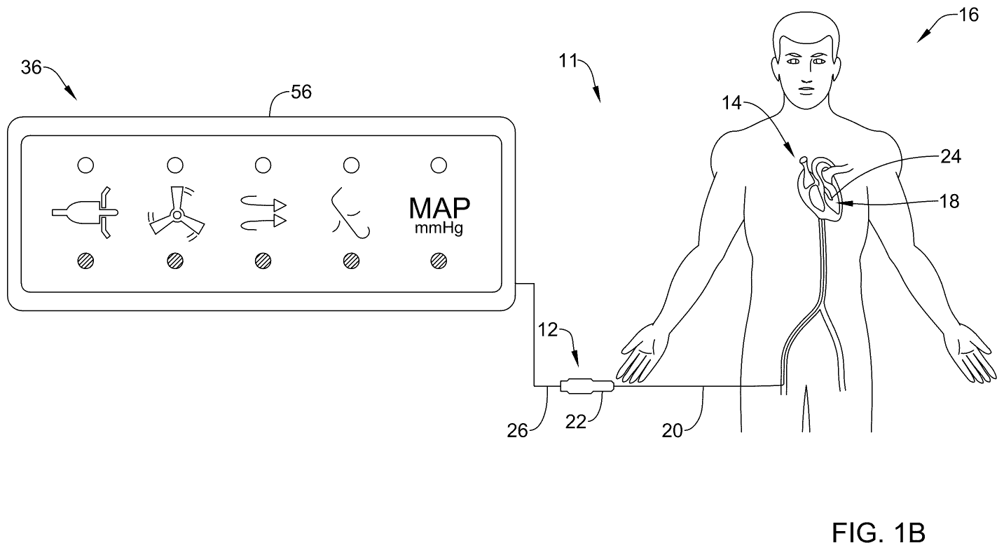

FIG. 1A depicts an illustrative percutaneous circulatory support system 10 including a circulatory support device 12 positioned in the heart 14 of a patient 16. The circulatory support device 12 may include, among other components, a flexible elongate catheter shaft 20 having a first end attached to a handle 22 and a second end attached to a blood pump 24. As depicted in FIG. 1A, the blood pump 24 may be positioned in the left ventricle 18 of the patient 16. The blood pump 24 may be delivered (e.g., tracked) to the left ventricle 18 percutaneously over a guidewire. For example, the catheter shaft 20 and the blood pump 24 may be tracked over a guidewire through the femoral artery and the descending aorta, over the aortic arch, through the ascending aorta, past the aortic valve, and into the left ventricle 18.

In some examples, the blood pump 24 may be positioned within the heart 14 such that a blood inlet positioned along the distal region of the blood pump 24 may be located in the left ventricle 18 and a blood outlet positioned along a housing of the blood pump 24 may be located in the aorta. Additionally, the blood pump 24 may include an electrically powered motor that drives rotation of an impeller (e.g., where the motor and/or the impeller may be positioned within the housing of the blood pump 24). In some examples, the motor may power the rotation of the impeller via electromagnetic induction, but other suitable configurations are contemplated. The rotating impeller may draw blood from the left ventricle (via the blood inlet) into the aorta (via the blood outlet). In other words, an electrically powered motor may drive the impeller to pump blood from the left ventricle through the aortic valve and into the ascending aorta.

FIG. 1A further illustrates that a first end of the catheter shaft 20 may be attached to a housing 22. The housing 22 may be considered a connector and include a distal end region attached to the catheter shaft 20 and a proximal end region attached to a cable 26. It can be appreciated that the housing 22 may include one or more actuators (e.g., buttons, levers, dials, switches, etc.) designed to permit a clinician to control various functions of the blood pump 24. For example, a clinician may be able to control the speed of the motor and/or the impeller located in the blood pump 24 via actuation of an actuator located on the housing 22, in some instances.

The cable 26 may include a proximal end region directly connected to a removable controller 36, as described herein. That is, in contrast to other approaches such as those that may directly couple the cable 26 to the console 28, the approaches herein directly couple the cable 26 to the removable controller 36 which can, in turn, be removably coupled to the console 28 (e.g., can be removably disposed within a docking port of the console 28, removably attached to the top or side of the console 28, etc.). In removably coupling the removable controller 36 to the console 28, electrical connections between the removable controller 36 and the console 28 may be established, providing electrical communication between the console 28 and the blood pump 24, via the cable 26. This indirect coupling of the cable 26 to the console 28 can promote aspects herein, such as permitting the removable controller 36 to be readily coupled to or decoupled from the console 28 (e.g., mounted to/in the console 28, yet detached from the console 28), and yet retain the communicative coupling between the removable controller 36 and the blood pump 24 which may permit ease of transport of a patient and yet permit uninterrupted operation, monitoring and/or control of the blood pump 24. The cable 26 may include or be an electrical power cable configured to power the blood pump 24, a communication cable configured to communicate data between the blood pump 24 and the console 28, an electrical cable, an optical cable, and/or other suitable type of cable.

The percutaneous circulatory support system 10 may include a sensor positioned within and/or along the console 28, the handle 22, the catheter shaft 20, and/or the blood pump 24, where the sensor may be coupled with the console 28 and/or other data monitoring component via wired connection (e.g., an electrical and/or optical connection of the cable 26) and/or a wireless connection. The sensor positioned within and/or along the console 28, within the handle 22, the catheter shaft 20, and/or the blood pump 24 may be configured to sense a parameter of or related to operation of the circulatory support system 10, operation of the circulatory support device 12, and/or the patient 16. The sensor positioned within and/or along the console 28, the handle 22, along the catheter shaft 20, and/or the blood pump 24 may be designed or configured to sense and/or monitor any suitable parameters including, but not limited to, blood pressures (e.g., arterial pressure, venous pressure), blood velocity, blood flowrate, mean arterial pressure, impeller speeds, motor speeds, an electrical current provided to the motor, voltage provided to the motor, back-EMF from the motor, and/or other suitable parameters, along with any suitable combination or temporal pattern of signals corresponding to the parameters. Further, additional parameters (e.g., flow through or across the blood pump 24) may be derived by processing combinations of sensed data in a time dependent manner.

As depicted in FIG. 1A, the console 28 may include controls (e.g., buttons, knobs, dials, touch-sensitive areas, etc.) 30 and/or one or more displays 31. FIG. 1A depicts the console 28 with a first display 32 and a second display 34, but other suitable configurations of the console 28 with a single display and/or with more than two displays are contemplated. An example circulatory support system including a console with two displays is disclosed in Appl. No. 63/457,935, filed on Apr. 7, 2023, and titled CIRCULATORY SUPPORT DEVICE SYSTEM, which is hereby incorporated by reference in its entirety for any and all purposes.

Additionally, while FIG. 1A illustrates the first display 32 and the second display 34 as being integrated into the console 28, it is contemplated that the circulatory support system 10 may be designed such that the first display 32, the second display 34, or both of the first display 32 and the second display 34 are separate, distinct components of the circulatory support system 10. In other words, the first display 32, the second display 34, or both of the first display 32 and the second display 34 may be separate stand-alone displays, apart from the console 28 (e.g., may instead be manifested as the display 52 in the removable controller when the removable controller 36 is removably coupled to the console 28, as described herein). In some examples, the first display 32 and the second display 34 may receive data from the same or different sources.

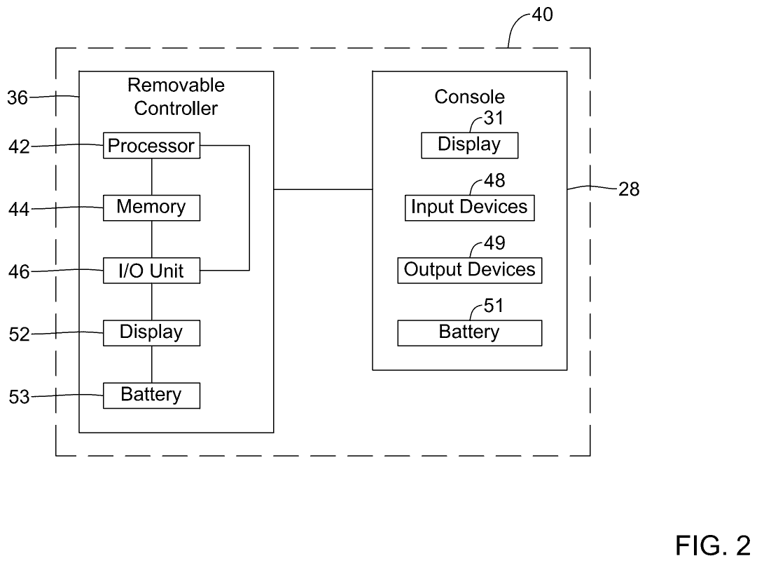

As detailed herein, in some instances the first display 32, the second display 34, or both of the first display 32 and the second display 34 may be separate components apart from the console 28 that are included in a removable controller 36. The removable controller 36 can be analogous to the computing device or removable controller 36 described herein with respect to FIG. 2.

As detailed herein, the removable controller 36 can be configured to control operation of the blood pump 24 in the absence of the console 28, and can be removably coupled to a console 28. For instance, the removable controller 36 can be configured to control operation of the blood pump 24 alone (e.g., when decoupled from the console 28) and may thereby promote aspects herein promoting ease of transporting a patient (in which the blood pump 24 is disposed) via an emergency medical services (EMS) (e.g., via helicopter, ambulance, etc.) and/or transporting the patient between locations within a medical facility, such as a hospital, (e.g., between a catheter laboratory, an operating room, an intensive care unit, and/or other location within the medical facility), and yet permit control and monitoring of the operation of the blood pump 24. For example, the removable controller 36 may be configured to couple to a belt, harness, sling, etc., and/or otherwise be coupled to the patient and thereby promote transportation and/or location of the patient, as compared to other approaches which require the presence of a bulky console (e.g., a console including or coupled to an elongate stand 13 (e.g., on top of which the console is disposed) and/or the console 28 including an external power supply 17, etc.).

For instance, FIG. 1B is a schematic diagram of another illustrative percutaneous circulatory support system 11 with a blood pump positioned in a patient. The percutaneous circulatory support system 11 is analogous to the percutaneous circulatory support system 10 in FIG. 1A, but with the change that the removable controller 36 has been removed or decoupled from the console 28 and the console 28 is no longer present (e.g., is no longer coupled to the patient). For instance, in some embodiments the removable controller 36 may include a battery (e.g., a rechargeable battery), which as detailed herein, may promote ease of transporting of the patient in the absence of requiring the patient to remain coupled to a power supply or other external power circuitry configured to power the bulky console 28. That is, the console 28 may be removably coupled to a removable controller 36, as described herein. The removable controller 36 can include a user interface such as a display that is separate from a user interface retained with the console 28 and which is permanently located in the console 28. For instance, FIG. 2 depicts a schematic box diagram of an illustrative computing device or removable controller 36 and console 28. The computing device or removable controller 36 and/or the console 28 may be entirely or partially housed in a housing 40. For instance, the console 28 may be permanently housed in the housing 40 and the computing device or removable controller 36 may be removably housed in the housing 40 and/or mounted on the housing 40. The housing 40 may be an optional component, as represented by the broken lines defining the housing 40 depicted in FIG. 2. Although various components are depicted as being included in the computing device or removable controller 36 and the console 28, one more of the depicted components may be omitted and/or one or more additional or alternative components may be utilized.

The computing device or removable controller 36 may be any suitable computing device configured to process data of or for the percutaneous circulatory support systems 10, 11 and may be configured to facilitate operation of the percutaneous circulatory support systems 10, 11. The computing device or removable controller 36, in some cases, may be configured to control operation of the blood pump 24 by establishing and/or outputting control signals to the blood pump 24 and/or components of or in communication with the console 28 to control and/or monitor operation of the blood pump 24. In some examples, the removable controller 36 may be removably coupled to the console 28 and communicate with the blood pump 24 over a wired and/or wireless connection, but other suitable configurations are contemplated.

In some cases, the removable controller 36 and/or the console 28 may communicate with a remote server or other suitable computing device. The entire removable controller 36, or at least a part of the removable controller 36, is a component separate from a structure of the console 28, that may communicate with electronic components of the blood pump 24, the console 28, and/or other suitable components of the percutaneous circulatory support systems 10, 11 over one or more wired or wireless connections or networks (e.g., LANs and/or WANs).

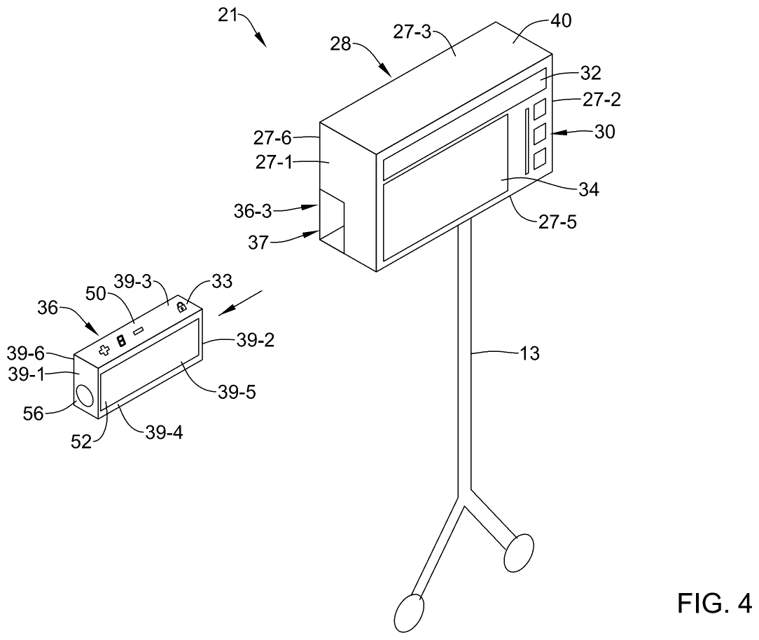

The removable controller 36 may include, among other suitable components, a processor 42 (e.g., a hardware processor), a memory 44 (e.g., a medium storing non-transitory instructions thereon), an I/O unit 46, a display 52, and a battery 53. Example other suitable components of the removable controller 36 that are not specifically depicted in FIG. 2 may include, but are not limited to, communication components, a touch screen, selectable buttons, wireless and/or wired communications components, power transmitting/and/or receiving components, and/or other suitable components of a removable controller. For instance, the removable controller 36 can include communications components such that the removable controller 36 is configured to be in wired communication, wired communication, or both, with the console 28. For example, the removable controller 36 can be in wired and/or wireless communication with the console 28 when the removable controller 36 is disposed in the docking port 37 (see FIG. 4). In other words, an electrical connection may be established between the removable controller 36 and the console 28 when the removable controller 36 is coupled to (e.g., docked with) the console 28, and the electrical connection may be broken between the removable controller 36 and the console 28 when the removable controller 36 is decoupled from (e.g., undocked with) the console 28.

In some instances, the removable controller 36 can be in wireless communication with the console 28 when the removable controller 36 is removed from the docking port 37 and remains proximate to the console 28 (e.g., is located on a common wireless network such as a common WiFi network as the console 28). In such instances, the patient may be able to periodically walk or otherwise be moved a distance away from the console 28 and yet remain in communication with the console 28. In an event in which the wireless communication with the console 28 is lost, the removable controller 36 may continue to operate the blood pump 24 uninterruptedly.

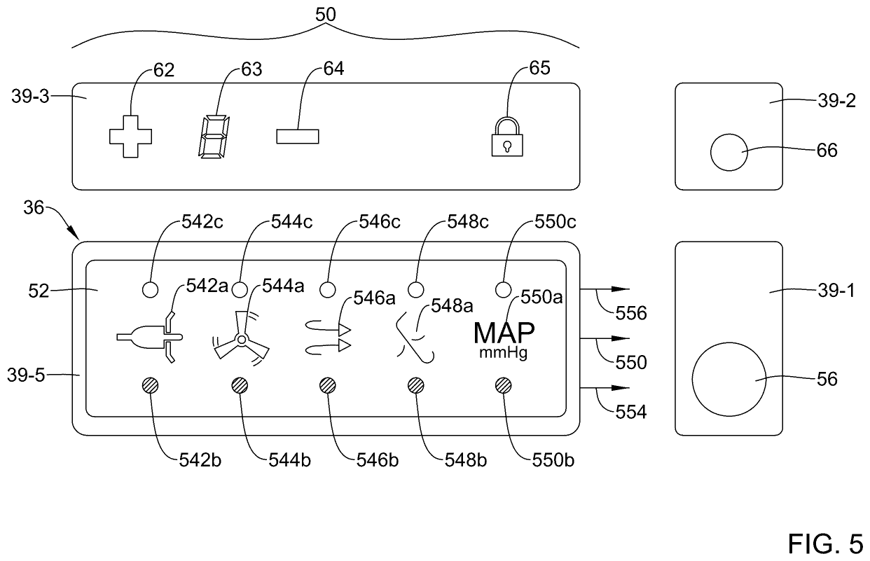

In some embodiments the removable controller 36 includes a battery 53 (e.g., a first battery). The battery 53 may be a rechargeable battery or a non-rechargeable battery. In some embodiments, the battery 53 is a recharge battery that is configured to be recharged in a wired and/or wireless manner. Stated differently, in some embodiments, the removable controller 36 can be configured to receive wireless power, wired power, or both, to charge the battery 53. For instance, the battery 53 can be a rechargeable battery that is configured to be recharged in a wired manner when the removable controller 36 is coupled to (e.g., docked with) the console 28 (e.g., is disposed in the docking port of the console 28) and/or a charger that is separate from the console 28. For instance, the battery 53 may receive wired power via a (e.g., the second connector 66 as illustrated in FIG. 5) and a corresponding connector (not illustrated) that is located in the docking port 37 of the console 28.

In some examples, the console 28 can include a battery 51 (e.g., a second battery) separate from the battery 53 of the removable controller 36. For instance, the battery 51 may be rechargeable battery or a non-rechargeable battery configured to supply power to the console 28 in the event of an unexpected loss of power to the console 28.

The processor 42 of the removable controller 36 may include a single processor or more than one processor working individually or with one another. The processor 42 may be configured to receive and execute instructions, including instructions that may be loaded into the memory 44 and/or other suitable memory. Example components of the processor 42 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 44 of the removable controller 36 may include a single memory component or more than one memory component each working individually or with one another. Example types of memory 44 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 44 may be or may include a non-transitory computer readable medium. The memory 44 may include instructions stored in a transitory state and/or a non-transitory state on a computer readable medium that may be executable by the processor 42 to cause the processor 42 to perform one or more of the methods and/or techniques described herein. Further, in some cases, the memory 44 and/or other suitable memory may store data received from the blood pump 24.

The I/O unit 46 of the removable controller 36 may include a single I/O component or more than one I/O component each working individually or with one another. Example I/O unit 46 may be or may include any suitable types of communication hardware and/or software including, but not limited to, communication components or ports configured to communicate with electronic components of the percutaneous circulatory support systems 10, 11, and/or with other suitable computing devices or systems. Example types of I/O unit 46 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units 46.

The console 28 may be configured to communicate with the computing device or removable controller 36 via a wired and/or a wireless connection. The console 28 may include a display 31 (e.g., such as the first display 32 and/or the second display 34, illustrated in FIG. 1A), an input device 48, an output device 49, and/or one or more other suitable features.

The display 31 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 48 may be and/or may include any suitable components and/or features for receiving user input via the console 28. Example input device(s) 48 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 48 may include a touch screen that allows for setting set points and/or selecting selectable elements for additional detail concerning data or information associated with the selectable elements, but this is not required.

The output device(s) 49 may be and/or may include any suitable components and/or features for providing information and/or data to users and/or other computing components. Example output device(s) 49 include, but are not limited to, displays, speakers, vibration systems, tactile feedback systems, optical outputs, and/or other suitable output devices. In some instances, the output device(s) 49 and the input device(s) may be the same device such as a display.

The removable controller 36 of the percutaneous circulatory support system 10 may be configured to facilitate control of the blood pump 24 and store and/or monitor data related to operation of the blood pump 24 (e.g., data from operation of the blood pump 24, data from control of the blood pump 24, data from sensors of and/or associated with the blood pump 24, and/or data from other suitable sources). The removable controller 36 may receive data over time from the blood pump 24 operating within a patient, where the received data may include patient data and/or data related to operation of the blood pump within the patient.

The removable controller 36 may be configured (e.g., sized and shaped) to be coupled to the blood pump 24 in a manner that allows for transporting the removable controller 36 (e.g., in the absence of the console 28) with the patient in which the blood pump 24 has been positioned and currently operating, which may include transporting the patient via an emergency medical services (EMS) (e.g., via helicopter, ambulance, etc.) and/or transporting the patient between locations within a medical facility, such as a hospital, (e.g., between a catheter laboratory, an operating room, an intensive care unit, and/or other location within the medical facility). To assess the patient and/or operation of the blood pump 24, medical professionals may need to evaluate historical patient data and/or blood pump 24 data gathered and/or saved at or by the removable controller 36.

To facilitate transportation of the removable controller 36 with the patient, the removable controller 36 may be lightweight and/or compact. However, it may be desirable for the console 28 to have a large display configured to facilitate displaying patient data and/or blood pump data that facilitates medical professionals assessing the patient. That is, the system and approaches herein yield both a lightweight and/or compact removable controller 36 that is a readily transportable (e.g., when removed from the console 28) to continue to operate the blood pump 24, and yet provide the console 28 with a large display configured to facilitate displaying patient data and/or blood pump data that facilitates medical professionals readily assessing the patient once transported to a particular location (e.g., a particular room in a hospital).

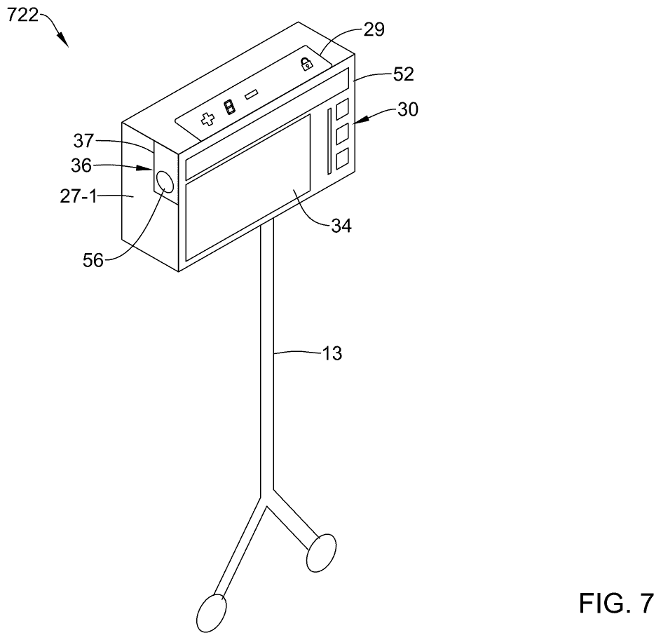

Additionally, in some instances, the console 28 may include a first display, which may be configured to display a screen providing limited data or information (e.g., preconfigured screens with fixed layouts), and the removable controller 36 may include a second display (e.g., the display 52) which may display screens having repetitive, condensed, or augmented views relative to what is depicted on the first display of the console 28. Such a configuration of the displays and screens for the displays may allow for reducing a cost and/or size of the console 28 relative to when two integral (permanent) displays are required on the console 28 and/or when a large single display is included on the console 28. An example of such a configuration is described in greater detail with respect to FIG. 7. Although the two-display configuration is discussed herein with respect to percutaneous circulatory support systems, similar concepts may be applied to other medical systems that include a computing device with limited display space, such as medical systems configured to be transported with a patient, medical systems for use in operating rooms with limited space, and/or other suitable medical systems.

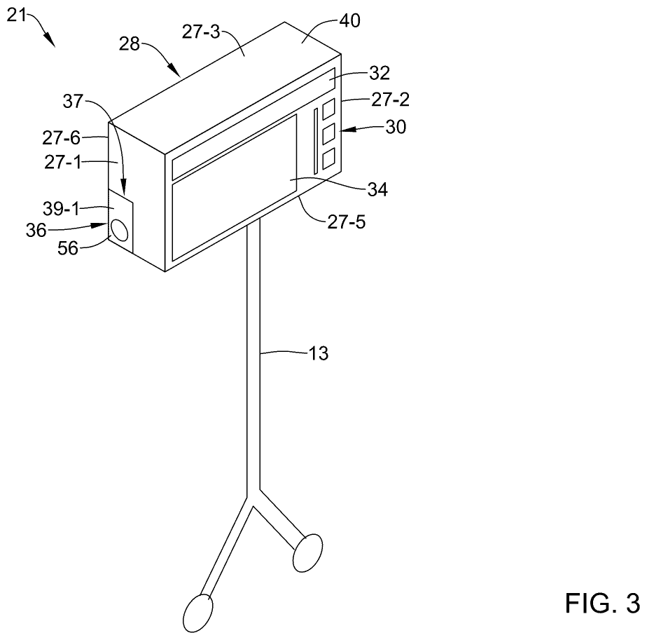

FIG. 3 is a schematic diagram of a portion of an illustrative circulatory support system 21 with a removable controller 36 coupled to (i.e., docked with) a console 28, while FIG. 4 is a schematic diagram of a portion of an illustrative circulatory support system 21 with the removable controller 36 decoupled from (i.e., undocked from) the console 28. As described herein, a blood pump (not illustrated in FIGS. 3-4) may be coupled to, and remain coupled to, the removable controller 36 (e.g., electrically coupled via the cable 26) when the blood pump is coupled to and/or is decoupled from the console 28. As such, the blood pump and the removable controller 36 may facilitate transportation and/or locomotion of a patient, and yet the blood pump may remain communicatively coupled to the removable controller 36 such that the blood pump may remain in uninterrupted operation. For instance, the removable controller 36 is configured at least due to the location of the first connector 56 and/or the second connector (e.g., the second connector 66 as illustrated in FIG. 5) and/or the inclusion of a battery or another portable power source in the removable controller 36 that is separate from a power source in the console 28 to remain communicatively coupled to the blood pump when the removable controller 36 is removably coupled to and/or is decoupled from the console 28. Accordingly, the blood pump may remain communicatively coupled to the removable controller 36 when the removable controller 36 is decoupled from or is coupled to the console 28, thereby permitting uninterrupted monitoring and/or control over operation of the blood pump, in contrast to other approaches that may, at least temporarily, disconnect a blood pump from a controller prior to, during, and/or subsequent to transportation (e.g., transportation in an ambulance) and/or locomotion of a patient (e.g., when a patient wishes to walk around or outside of a hospital room or other room in which they are primarily staying).

The console 28 and the removable controller 36 are analogous to the console 28 and the removable controller 36 discussed herein (e.g., discussed previously with respect to FIGS. 1A, 1B, and 2). For instance, the console 28 can include controls 30, a first display 32 and a second display 34, as illustrated in FIG. 3.

The console 28 can include a housing 40. The housing 40 can include a plurality of surfaces such as a first surface 27-1, a second surface 27-2, a third surface 27-3, a fourth surface 27-4, a fifth surface 27-5, and a sixth surface 27-6. The first surface 27-1 can be a top surface of the housing 40 and the second surface 27-2 can be a bottom surface of the housing 40 that is opposite the first surface 27-1. The third surface 27-3 can be a first side surface of the housing 40 and the fourth surface 27-4 can be a second side surface of the housing 40 that is opposite the third surface 27-3. The fifth surface 27-5 can be a front surface of the housing 40 and a sixth surface 27-6 can be a back surface of the housing 40 that is opposite fifth surface 27-5.

Similarly, the removable controller 36 can include a housing 33. The housing 33 can be separable from the housing 40 of the console 28. The housing 33 can include a plurality of surfaces such as a first surface 39-1, a second surface 39-2, a third surface 39-3, a fourth surface 39-4, a fifth surface 39-5, and a sixth surface 39-6. The first surface 39-1 can be a top surface of the housing 33 and the second surface 39-2 can be a bottom surface of the housing 33 that is opposite the first surface 39-1. The third surface 39-3 can be a first side surface of the housing 33 and the fourth surface 39-4 can be a second side surface of the housing 33 that is opposite the third surface 39-3. The fifth surface 39-5 can be a front surface of the housing 33 and a sixth surface 39-6 can be a back surface of the housing 33 that is opposite fifth surface 39-5.

As illustrated in FIG. 3, the console 28 can include the controls 30, the first display 32 and the second display 34 disposed on the fifth surface 27-5 (e.g., front surface) of the housing 40. However, other configurations where the controls 30, the first display 32 and/or the second display 34 are disposed on a different surface of the housing 40 and/or a surface of the housing 33 are possible. For instance, as described herein in some instances the first display 32, the second display 34, or both the first display 32 and the second display 34 can be replaced by a display on a surface of the removable controller 36. Similarly, in some examples the control 30 on the console 28 may be replaced by controls located on a surface of the removable controller 36. Stated differently, in some embodiments, the console 28 may not include controls 30 and may instead be configured to removably coupled to the removable controller 36 which includes controls (e.g., the controls 50 such as speed controls and/or speed lock controls, etc.).

As illustrated in FIG. 3, the console 28 can include a docking port 37. The docking port 37 refers to a cavity formed within a surface of the console 28 or a receptacle formed on a surface of the console 28. For instance, as illustrated in FIG. 3, the docking port 37 can be formed in a side surface such as the first surface 27-1 of the console 28.

In some embodiments, the removable controller 36 and the docking port 37 can be configured (e.g., sized and shaped) such that the removable controller 36 is disposed at least partially within the cavity of the docking port 37 of the console 28. For instance, the removable controller 36 can have substantially the same shape as the cavity of the docking port 37, as illustrated in FIG. 4. For instance, the docking port 37 can be a substantially rectangular shaped docking port configured to receive a substantially rectangular shaped removable controller 36, as illustrated in FIGS. 3-4. Having the removable controller 36 be disposed at least partially within the docking port 37 can promote aspects herein such as securely and removably coupling the removable controller 36 to the docking port 37. For instance, as illustrated in FIG. 3, the removable controller 36 can be configured such that the removable controller 36 is disposed entirely within the docking port 37 of the console 28 when the removable controller 36 is removably coupled to the console 28.

The docking port 37 can include a corresponding connector (not shown) that is configured to couple to a connector (e.g., a second connector) in the removable controller 36. For instance, the docking port 37 may be a substantially rectangular shaped docking port having a corresponding connector configured couple to a connector (e.g., the second connector 66 on the second surface 39-2, as illustrated in FIG. 5) in the removable controller 36. That is, the corresponding connector in the docking port 37 can be coupled to a connector (e.g., the second connector 66, as illustrated in FIG. 5) to communicatively couple the removable controller 36 with the console 28. The removable controller 36 and the console 28 may exchange information and/or power when communicatively coupled. For instance, when communicatively coupled the removable controller 36 and/or the console 28 may be configured to control and/or otherwise communicate information with the blood pump and/or the console 28.

In some instances, the docking port 37 can extend from an outer surface of the console 28 into a cavity of the console 28. For instance, the docking port 37 can extend into the first surface 27-1 and be located proximate to both the fourth surface 27-4 (e.g., the bottom surface) and the sixth surface 27-6 (e.g., the back surface 27-6), as illustrated in FIGS. 3-4. Having the docking port 37 located in a side surface and be located proximate to both the fourth surface 27-4 (e.g., the bottom surface) and the sixth surface 27-6 (e.g., the back surface), as illustrated in FIGS. 3-4, can promote aspects herein such as protecting a display 52 of the removable controller 36 when the removable controller 36 is disposed in the docking port 37, and permitting access to the first connector 56 when the removable controller 36 is disposed in the docking port 37. For instance, the docking port 37 may be configured to obscure (e.g., protect) at least the display 52 of the removable controller 36 when the removable controller 36 is disposed in the docking port 37. For example, as illustrated in FIG. 3, the docking port 37 may be configured to obscure both the controls (e.g., controls 50 as illustrated in FIG. 5) and the display 52 and thereby protect the controls and the display 52 from unintended contact. In such embodiments, the removable controller 36 may be configured to cease operation (e.g., cease registering inputs from and/or cease/reduce power provided to the speed controls and/or the display 52. Ceasing operation of the controls and/or the display 52 may reduce power consumption of the removable controller 36 and/or may mitigate any unintended input received via the controls and/or the display 52 while the removable controller 36 is disposed in the docking port 37, and thereby may enhance operation of the percutaneous circulatory support system 10. For instance, in some embodiments, the removable controller 36 can be configured to cease display of status indicators when the removable controller 36 is removably coupled to the docking port 37 of the console 28.

However, the docking port 37 may be located in/on and/or proximate to another surface of the console. For instance, the docking port 37 may be located in the first surface 27-1 of the console 28 and can be located proximate to at least the fifth surface 27-5 (e.g., a front surface) of the console 28, as described herein. Having the docking port 37 be located in a side surface such as the first surface 27-1 of the console 28 and also be located proximate to at least the fifth surface 27-5 of the console can promote aspects herein such as permitting, the controls (e.g., the controls 50 as illustrated in FIG. 5) and/or the display 52 of the removable controller 36 to be accessible when the removable controller 36 is disposed in the docking port 37. For instance, FIG. 7 describes an example where both the controls (e.g., the speed controls) and the display 52 are accessible and remain enabled when the removable controller 36 is disposed in or otherwise docked with the console 28 via the docking port 37, as detailed herein. That is, in some instances, the docking port 37 is configured to expose at least the display 52 of the removable controller 36 when the removable controller 36 is disposed in or otherwise docked with the console 28 via the docking port 37. In such instances, the removable controller 36 can be configured to continue the display of status indicators (e.g., the second set of status indicators) when the removable controller 36 is removably coupled to the console 28. In such instances, the removable controller 36 can be configured to continue providing power to and receiving signals from the speed controls (e.g., speed controls 62, 64 as illustrated in FIG. 5). For instance, the removable controller 36 may be configured to control the speed of the blood pump at least with the controls 50. For instance, in some examples, the controls 50 can include speed controls that are configured to control the speed of the blood pump when the removable controller 36 is coupled to and when the removable controller 36 is decoupled from the docking port 37 of the console 28. In such instances, the console 28 may be without dedicated speed controls therein, which may reduce a size and/or cost of the console 28. However, in some instances the console 28 may include dedicated speed controls 30, as illustrated in FIGS. 3-4, operational to control the speed of the blood pump when the removable controller 36 is docked with the console 28.

In some embodiments, the docking port 37 and the removable controller 36 can be configured such that a surface of the docking port 37 is coplanar with an adjacent surface of the removable controller 36 when the removable controller 36 is disposed in the docking port 37. Alternatively or in addition, the first surface 39-1 of the removable controller 36 can be coplanar with an outer surface of the console 28 when the removable controller 36 is disposed entirely within the docking port 37 of the console 28. For example, the first surface 39-1 of the removable controller 36 can be coplanar with the first surface 27-1 of the console 28. Having at least one outer surface of the removable controller 36 be coplanar with at least one outer surface of the console 28 can promote aspects herein such as minimizing a size of the console 28 and/or protecting the removable controller 36.

In some embodiments, a portion of the removable controller 36 may be visible and physically accessible when the removable controller 36 is disposed in or otherwise docked with the docking port 37. For instance, at least one surface of the removable controller 36 may be visible and physically accessible from exterior of the console 28 when the removable controller 36 is disposed in or otherwise docked with the docking port 37. Having at least one surface of the removable controller 36 be visible and physically accessible can promote aspects herein such as permitting a blood pump (not illustrated in FIGS. 3-4) to remain coupled to a connector (e.g., the first connector 56) located on the visible and physically accessible surface of the removable controller 36.

FIG. 5 is a schematic diagram showing a plurality of surfaces of the removable controller 36. As illustrated in FIG. 5, the plurality of surfaces of the removable controller 36 can include those surfaces previously discussed such as the first surface 39-1, the second surface 39-2, the third surface 39-3, and the fifth surface 39-5.

The first surface 39-1 (e.g., the first side surface) can include the first connector 56. As mentioned, the first connector 56 can be configured to communicatively couple the removable controller 36 with the blood pump via the cable 26.

The second surface 39-2 (e.g., the second side surface) can include the second connector 66. As mentioned, the second connector 66, can be configured to communicatively couple the removable controller 36 with the console 28. For instance, the second connector 66 can be configured to communicatively couple the removable controller 36 to the console 28 when the removable controller 36 is disposed in or otherwise docked with a docking port of the console 28, as described herein.

The first connector 56 and/or the second connector 66 may be connectors manifested as ports and/or protrusions. For instance, as illustrated in FIG. 5, the first connector 56 and the second connector 66 can each be ports. Having both the first connector 56 and the second connector 66 be formed as a respective port may promote aspects herein such as providing a removable controller 36 with generally planar outer surfaces (e.g., without protruding connectors) and thereby minimizing a propensity for the removable controller 36 to snag on or otherwise inadvertently interact with the patient and/or other objects when the removable controller is decoupled from the console 28. However, the use of other types of connectors is possible.

The third surface 39-3 (e.g., the top surface) can include controls 50 such as the speed controls 62, 64 and/or a speed lock 65. The speed control 62 can increase a speed of a blood pump coupled via the first connector 56 to the removable controller 36. The speed control 64 can decrease a speed of the blood pump. The speed lock 65 may be configured to lock the blood pump at a given speed and thereby negate any inputs to the speed controls 62, 64 when the speed lock is in a locked position. The speed lock 65 may be configured to permit alteration of a speed of the blood pump via an input to the speed controls 62, 64 when the speed lock is in an unlock position. The speed indicator 63 can include a current speed and/or speed level of the blood pump.

The fifth surface 39-5 (e.g., the front surface) can include a display 52. The display 52 can be similar in form and function to other displays discussed herein. The display can include visual parameter representations 542a, 544a, 546a, 548a, 550a which are horizontally aligned with one another along the display 52 along the horizontal axis 550. FIG. 5 further illustrates that each of the visual parameter representations 542a, 544a, 546a, 548a, 550a may include a symbol (e.g., icon) which may represent a particular functional or operational parameter of the percutaneous circulatory support systems 10, 11 or a physiological parameter of the patient 16.

As illustrated in FIG. 5, the first visual representation 542a corresponds to a status of an electrical connection between the cable 26 (e.g. the electrical cable) and controller 36/console 28, the second visual representation 544a corresponds to a status of a motor rotating within the blood pump, the third visual representation 546a corresponds to a status of blood flow (e.g., in terms of a pump flow rate or blood flow rate through the blood pump), the fourth visual representation 548a corresponds to a status of position of the blood pump within a vasculature of the patient 16, and the fifth visual representation 550a corresponds to values of mean arterial pressure (e.g., 99 mmHg), a blood pressure, an aortic pressure (AO), a left ventricular pressure (LV), and/or other suitable pressure or pressure related values. The parameter values may be updated at any suitable interval including, but not limited to, every beat of the patient's heart, every second, every minute, every five minutes, every thirty minutes, and/or over other suitable periods of time.

Alternatively or in addition, different parameters associated with the percutaneous circulatory support system 10 and/or the patient 16 may be represented on the display 52. For instance, indicators of a status (e.g., a current percentage charge) of the battery in the removable controller 36 may be displayed. The visual representations can be any suitable information related to the patient, the removably controller 36, and/or operation of the blood pump 24. In some examples, the visual representation may be based on live information related to the patient and/or operation of the blood pump 24. That is, the removable controller 36 can be configured to provide real-time or near real-time indications of a status of the patient and/or operation of the blood pump 24.

Each of the visual parameter representations can have a warning and/or alarm status indicator associated therewith. For instance, as illustrated in FIG. 5, each of the visual parameter representations can have two respective visual parameter representations associated therewith. For example, FIG. 5 illustrates the first-level warning and/or alarm status indicators 542b, 544b, 546b, 548b, 550b (e.g., cautionary warning and/or alarm) represented by a circle, whereby each of the circle-shaped status indicators 542b, 544b, 546b, 548b, 550b are aligned with one another along the horizontal axis 554. Further, FIG. 5 illustrates the second-level warning and/or alarm status indicators 542c, 544c, 546c, 548c, 550c (e.g., critical warning and/or alarm) represented by a circle, whereby each of the circle-shaped status indicators 542c, 544c, 546c, 548c, 550c are aligned with one another along the horizontal axis 556. FIG. 5 illustrates that the first-level warning and/or alarm status indicators 542b, 544b, 546b, 548b, 550b, the second-level warning and/or alarm status indicators 542c, 544c, 546c, 548c, 550c shown on display 52 may be vertically aligned with their corresponding visual parameter representations 542a, 544a, 546a, 548a, 550a, respectively, along a given vertical axis (e.g., the first-level warning and/or alarm may be located below the corresponding visual parameter representation and the second-level warning and/or alarm may be located above the corresponding visual parameter representation). However, other alignments are possible. As described herein, any of the status indicators (or visual parameter representations) illustrated in FIG. 5 may be represented by any shape, including a square, triangle, rectangle, oval, circle, star, polygon, etc.

As discussed herein, each of the visual parameter representations 542a, 544a, 546a, 548a, 550a illustrated and described herein, may identify (e.g., represent, symbolize, etc.) a particular functional or operational parameter of the percutaneous circulatory support system 10 or a physiological parameter the patient 16, while also conveying information relating to the “status” of that particular parameter during a medical procedure. For example, the display 52 may be configured to present status information relating to a functional parameter of the percutaneous circulatory support systems 10, 11 or a physiological parameter the patient 16 in a simple, easy-to-understand format such that a clinician my easily determine from the display 52 the status of each parameter represented by the visual parameter representations 542a, 544a, 546a, 548a, 550a. For instance, the visual parameter representations 542a, 544a, 546a, 548a, 550a may be illuminated during normal (e.g., routine) operation in which the associated parameter is functioning normally. For example, the first visual representation 542a may be illuminated (e.g., white light) when the electrical connection between the cable 26 (e.g. the electrical cable) and controller 36/console 28 is established, the second visual representation 544a may be illuminated (e.g., white light) when the motor is rotating normally, the third visual representation 546a may be illuminated (e.g., white light) when normal blood flow is detected through the blood pump, the fourth visual representation 548a may be illuminated (e.g., white light) when the system detects that the blood pump is correctly positioned across the aortic valve of the patient, and the fifth visual representation 550a may be illuminated (e.g., white light) when the sensed mean arterial pressure, or other a blood pressure, is within a normal range.

Accordingly, in some instances, the first-level and/or second-level warning and/or alarm status indicators of a given visual parameter representations may provide real-time, stepwise (e.g., progressive), status information (e.g., an alert indication) corresponding to an abnormal functional or a physiological parameter of the percutaneous circulatory support systems 10, 11 or patient 16.

Illumination of one or more of the first-level warning and/or alarm status indicators 542b, 544b, 546b, 548b, 550b is indicative of a cautionary warning and/or alarm associated with abnormal function of the percutaneous circulatory support systems 10, 11 and/or abnormal function or out of range physiological parameter of the percutaneous circulatory support systems 10, 11 or patient 16. Accordingly, in some instances the visual parameter representation 542a shown in FIG. 5 may include a symbol which conveys to a clinician that the visual parameter array 542a represents the normal electrical connection between the cable 26 and console 28 of the percutaneous circulatory support system 10. The display of the first-level warning and/or alarm status indicator 544b may convey that the status of the electrical connection between the cable 26 and console 28 is not in normal operating condition (e.g., an insufficient amount of current is being transferred between the console 28 and the circulatory support device 12 along the cable 26 and/or no electrical connection is established). In other words, displaying any of the first-level warning and/or alarm status indicators 542b, 544b, 546b, 548b, 550b may convey to a clinician that the parameter to which the first status indicator 542b, 544b, 546b, 548b, 550b relates is not operating normally and intervention is necessary.

Illumination of one or more of the second-level warning and/or alarm status indicators 542c, 544c, 546c, 548c, 550c is indicative of a serious or critical warning and/or alarm associated with abnormal function of the percutaneous circulatory support systems 10, 11 and/or abnormal function or out of range physiological parameter of the percutaneous circulatory support systems 10, 11 or patient 16. In some embodiments, a second-level warning and/or alarm status indicator such as the second-level warning and/or alarm status indicator 542c of the parameter represented by the visual parameter representation 542a (e.g., the electrical connection between the cable 26 and console 28) can be displayed. The second status indicator 542c may be configured to indicate that the visual parameter representation 542a has changed to a “serious” condition or “critical” condition which may indicate that the functional, operational or physiological parameter represented by the visual parameter representation 542a has moved out of the normal or operating range or a cautionary operating range and into a serious or critical condition, whereby user action maybe be required and the parameter may need improvement, adjustment, etc. (e.g., the electrical current being transferred between the console 28 and the circulatory support device 12 along the cable 26 may be in compromised condition).

Further, in some examples, the processing system of the percutaneous circulatory support systems 10, 11 may be programmed to define a range of values, minimum threshold, maximum threshold, etc. that defines the “serious” or “critical” operating condition of the particular parameter to which the given second-level warning and/or alarm status indicators 542c, 544c, 546c, 548c, 550c relate.

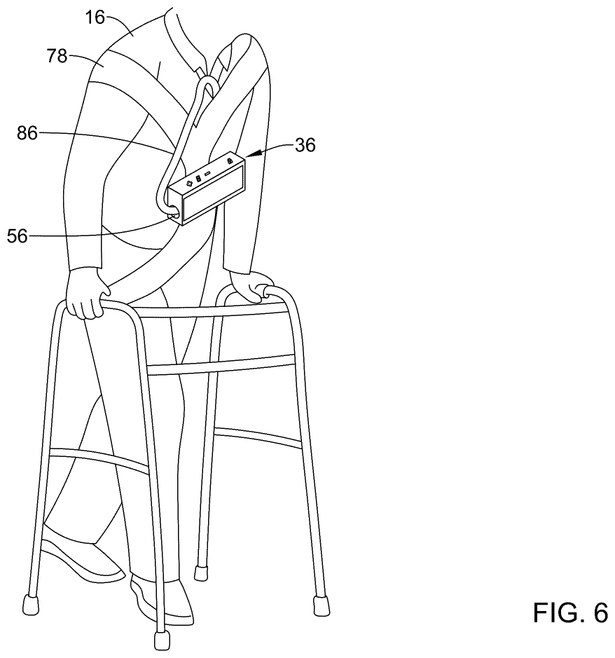

FIG. 6 is a schematic diagram showing a removable controller 36 coupled to a patient 16. As illustrated in FIG. 6, the removable controller 36 can be coupled to a wearable device 78 that can be worn externally by the patient 16. The wearable device 78 may be a belt, a harness, sling, or other type of apparatus that is configured to be worn externally by the patent 16 and receive the removable controller 36 during operation of the blood pump away from the console 28. For instance, the wearable device 78 may include a pouch or other cavity in which the removable controller 36 can be disposed and maintain a coupling with the blood pump when disposed therein. Alternatively or in addition, the wearable device 78 may include a mechanical coupling device such as flange or other type of mechanical component that is configured to interface (e.g., friction fit) with a corresponding mechanical component (e.g., a third connector) on the removable controller 36. For instance, the removable controller 36 can include a third connector (not illustrated) that is configured to be mechanically coupled the removable controller 36 to the wearable device 78 which is manifested as harness, as illustrated in FIG. 6. The removable controller 36 may be configured to be mechanically coupled to the wearable device 78 while also being coupled via the cable 26 to a blood pump inside the patient 16. Having the removable controller 36 be decoupled from a console and be coupled to the wearable device 78 can promote aspects herein such as permitting live control and/or monitoring (e.g., via the display 52) of a blood pump disposed inside the patient 16 and yet permitting ease of transport of the patient 16. For instance, the removable controller 36 can be communicatively coupled to the blood pump via a cable 86 when the removable controller is coupled to and when the removable controller 36 is decoupled from a console, as described herein. Cable 86 may be analogous to or similar to cable 26 as described herein.

FIG. 7 is a schematic diagram of a portion of another illustrative circulatory support system 772 with a removable controller 36 coupled to or otherwise docked with a console 28. FIG. 7 is similar to FIG. 3, with the change that the docking port 37 (which extends into the first surface 27-1) is located adjacent to the third surface 27-3 (e.g., the top surface) and the fifth surface 27-5 (e.g., the front surface) of the console 28. There may be one or more windows (e.g., passages) extending through a portion of a surface of the console 28 to expose a portion of the removable controller 36 disposed in the console 28. For instance, the third surface 27-3 (top surface) of the console 28 can include a window 29 to expose the controls 50 of the removable controller 36 when the removable controller 36 is disposed in the docking port 37. Similarly, the fifth surface 27-5 (front surface) of the console 28 can include a window to expose the display 52. Thus, in some instances the controls 50 and the display 52 may be accessible when the removable controller 36 is disposed in the docking port 37.

In such instances, the controls 50 and the display 52 of the removable controller 36 can be configured to remain active when the removable controller 36 is disposed in the docking port 37. That is, the controls 50 may permit control of the pump speed, etc. and the display 52 may continue to display information associated with the blood pump and/or the patient when the removable controller 36 is disposed in the docking port 37. In some instances, the display 52 and/or the controls 50 may provide a degree of redundancy and thereby be available in the event of the same display and/or same controls in the console 28. However, in some instances the display 52 and/or the controls 50 may take the place of a display (e.g., display 32) and/or controls (e.g., controls 30) that typically would be employed in the console 28. For instance, the controls 50 may include the only speed controls in the percutaneous circulatory support system 10, in some instances. Having the display 52 and/or the controls 50 take the place of a display (e.g., display 32) and/or controls (e.g., controls 30) that typically would be employed in the console 28 may reduce a cost and/or size of the console 28.

In some instances, the display 52 may be configured to display at least a first set of status indicators corresponding to parameters related to operation of the blood pump. In such instances, another display in the console 28 such as the display 32 and/or the display 52, as illustrated in FIG. 4) may be configured to display a second set of status indicators corresponding to parameters related to operation of the blood pump. In some embodiments, the second set of status indicators can be the same as the first set of status indicators. For instance, the display 32 may be configured to a second set of status indicators corresponding to parameters related to operation of the blood pump that are the same (e.g., are each identical to) the first set of status indicators corresponding to the parameters related to operation of the blood pump display by the display 52.

However, in some instances, the display 52 can be configured to display at least a first set of status indicators corresponding to parameters related to operation of the blood pump that is different than a second set of status indicators corresponding to parameters related to operation of the blood pump displayed by another display. For instance, the display 52 can be configured to display at least a first set of status indicators corresponding to parameters related to operation of the blood pump that is different than a second set of status indicators corresponding to parameters related to operation of the blood pump displayed by the display 52.

The display 52 may have a first surface area with limited dimensions for displaying screens due to the portable nature of removable controller 36. The display 52 can have a surface area that is less than a surface area of a display in the console such as the display 54. The larger surface area of the console display 54 relative to the surface area of the display 52 of the removable controller 36 may allow for additional information and/or data to be provided or accessible to the user relative to the information and/or data provided on screens of the console display 54 and/or facilitate other aspects herein such as promoting ease of transport of the removable controller 36

To facilitate the surface area with limited dimensions and/or for other purposes, screens configured for display on the display 52 may have a preconfigured, fixed layout, may be limited in the data and/or information provided (e.g., displayed), and may provide the limited data and/or information in a clear manner that facilitates addressing current conditions of the patient and/or the blood pump 24. Example screens for display on the display 52 are described herein, but other suitable configurations of screens for display on the display 52 are contemplated.

In some configurations, the removable controller 36 can be configured to communicate with the console 28 directly through a wired or wireless connection. Additionally or alternatively, the removable controller 36 (e.g., a computing device of the removable controller 36 or a computing device encompassing the removable controller 36) may be configured to communicate with the console 28 through a server, a cloud computing system, and/or other suitable computing device(s).

In some instances, the console 28 may be configured to store data (e.g., live data, historical data, etc.) related to operation of the blood pump 24 (e.g., patient and/or blood pump data) and/or send the data related to operation of the blood pump 24 to a server (e.g., a server that is part of a LAN and/or a server that is part of a WAN). In some examples, the console 28 and/or the server may include non-volatile memory to facilitate storing data at the console 28 and/or the server.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A circulatory support system comprising:
a blood pump; and
a removable controller configured to control operation of the blood pump and be removably coupled to a console, the removable controller comprising:
a display configured to display status indicators corresponding to parameters related to operation of the blood pump;
speed controls configured to control a speed of the blood pump;
a first connector configured to communicatively couple the removable controller with the blood pump; and
a second connector configured to communicatively couple the removable controller with the console.

2. The system of claim 1, wherein:
the first connector is disposed at a first surface of the removable controller; and
the second connector is disposed at a second surface of the removable controller.

3. The system of claim 2, wherein the first surface is a first side surface and the second surface is a second side surface that is opposite the first side surface.

4. The system of claim 1, wherein the removable controller is configured to be disposed at least partially within a docking port of the console.

5. The system of claim 4, wherein the removable controller is configured to be disposed entirely within the docking port of the console.

6. The system of claim 5, wherein the first surface is coplanar with an outer surface of the console when the removable controller is disposed entirely within the docking port of the console.

7. The system of claim 1, wherein the removable controller is configured such that at least the first connector remains physically accessible when the removable controller is removably coupled to the console.

8. The system of claim 1, wherein at least the first connector and the display remain accessible when the removable controller is removably coupled to the console.

9. The system of claim 1, wherein the removable controller is configured to be in wired communication, wireless communication, or both, with the console.

10. The system of claim 1, wherein the removable controller includes a rechargeable battery.

11. The system of claim 10, wherein the removable controller is configured to receive wireless power, wired power, or both, to recharge the rechargeable battery.

12. The system of claim 1, including a wearable device configured to receive the removable controller, and wherein the removable controller is configured to be removably coupled to the wearable device.

13. The system of claim 12, wherein the removable controller includes a third connector disposed on an outer surface of the removable controller, and wherein the third connector is configured to removably couple the removable controller to the wearable device.

14. The system of claim 1, wherein the removable controller is configured to remain communicatively coupled to the blood pump when the removable controller is removably coupled to the console to operate the blood pump.

15. The system of claim 14, wherein the removable controller is configured to remain communicatively coupled to the blood pump when the removable controller is decoupled from the console to operate the blood pump.

16. A circulatory support system comprising:
a blood pump;
a console including:
a display configured to display at least a first set of status indicators corresponding to parameters related to operation of the blood pump; and
a docking port; and
a connector; and
a removable controller comprising:
a user interface configured to display a second set of status indicators corresponding to parameters related to operation of the blood pump;
speed controls configured to control a speed of the blood pump;
a first connector configured to communicatively couple the removable controller with the blood pump; and
a second connector configured to connect to the connector of the console when the removable controller is docked with the console via the docking port to communicatively couple the removable controller to the console.

17. The system of claim 16, wherein:
the docking port is configured to obscure at least the display of the removable controller when the removable controller docked with the console via the docking port; and
the removable controller is configured to cease display of the second set of status indicators when communicatively coupled to the console.

18. The system of claim 16, wherein:
the display of the removable controller remains exposed when the removable controller is docked with the console via the docking port; and
the removable controller is configured to continue to display the second set of status indicators when communicatively coupled to the console.

19. A circulatory support system comprising:
a blood pump;
a console including:
a display configured to display at least a first set of status indicators corresponding to parameters related to operation of the blood pump; and
a docking port;
a connector; and
a first battery; and
a removable controller comprising:
a display configured to display a second set of status indicators corresponding to parameters related to operation of the blood pump;
speed controls configured to control a speed of the blood pump;
a first connector configured to communicatively couple the removable controller with the blood pump;
a second connector configured to connect to the connector of the console when the removable controller is docked with the console via the docking port to communicatively couple the removable controller with the console; and
a second battery.

20. The system of claim 19, wherein:
the removable controller is configured to maintain operation of the blood pump when undocked from the console.
